(19) United States
(12) Patent Application Publication
(10) Pub. No.: US 2008/0026020 A1
(43) Pub. Date: Jan. 31, 2008

(54) Cosmetic composition combining a C-glycoside derivative and an associative polymer

(75) Inventors: Claudie Willemin, Paris (FR); Veronique Chevalier, Villecresnes (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/822,115

(22) Filed: Jul. 2, 2007

Related U.S. Application Data
(60) Provisional application No. 60/836,395, filed on Aug. 9, 2006.

(30) Foreign Application Priority Data
Jul. 3, 2006 (FR) 06 06021

(51) Int. Cl. A61K 8/72; A61K 31/70; A61K 8/02; A61Q 17/00; A61Q 5/00; A61Q 19/00; A61P 17/00; A61K 8/18

(52) U.S. Cl. 424/401; 424/59; 424/63; 514/25; 514/53; 514/54

(57) ABSTRACT

The present invention relates to a cosmetic and/or pharmaceutical composition, especially a dermatological composition, comprising, in a physiologically acceptable medium containing an aqueous medium, at least one C-glycoside derivative and at least one associative polymer.

This non provisional application claims the benefit of French Application No. 06 06021 filed on Jul. 3, 2006 and U.S. Provisional Application No. 60/836,395 filed on Aug. 9, 2006.

The present invention relates to cosmetic and/or dermatological compositions comprising a C-glycoside derivative and an associative polymer, and also to the use of these compositions in a process for treating keratin materials and/or fibres.

More particularly, the compositions of the invention are intended for caring for and/or making up keratin materials and/or fibres.

For the purposes of the invention, the term “keratin materials and/or fibres” is intended to denote, for example, the skin, mucous membranes, the lips, the scalp, the eyelashes, the eyebrows and the hair.

Sugars and sugar derivatives are products that have already been exploited for various purposes for the formulation of cosmetic compositions intended either for skincare or for caring for and/or washing keratin fibres.

Thus, in U.S. Pat. No. 6,495,147, D-xylose and derivatives thereof are proposed for the purposes of preparing cosmetic or pharmaceutical products aimed at improving the functionality of epidermal cells.

Among the sugars that may be used in the field, C-glycoside derivatives prove to be most particularly advantageous. Thus, certain C-glycoside derivatives have demonstrated advantageous biological properties, in particular for combating ageing of the epidermis and/or skin dryness. Such compounds are especially described in document U.S. Pat. No. 7,049,300.

These compounds are more particularly represented by the formula: 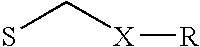
in which S represents a monosaccharide or a polysaccharide, R represents various linear or cyclic radicals and the group X may represent a group chosen from: —CO—, —CH(NR₁R₂)—, CHR′-, —C(═CHR′)- with R₁, R₂ and R′ possibly representing various radicals, including the hydroxyl radical for R₁ and R₂.

Moreover, it is also sought to have available cosmetic compositions that can be applied satisfactorily to keratin materials, and, to this end, these compositions should have textures suitable for allowing good application (especially good spreading) on keratin materials.

Unfortunately, the introduction of the C-glycoside derivatives mentioned above into an aqueous cosmetic formulation may be reflected by an appreciable reduction in the viscosity, thus giving rise to substantial fluidization of the composition.

An excessively fluid composition is difficult to apply to keratin materials. Such a composition runs on the keratin materials, especially on the skin, onto which it is applied. Its application to the keratin materials that it is desired to treat lacks precision and thus makes it relatively unattractive to use.

In addition, the presence of a C-glycoside derivative is found to affect the thickening power of certain conventional gelling agents.

Alternative solutions consisting in compensating for this reduction in viscosity by adding wax(es) and/or fatty alcohol do not prove to be satisfactory. The formulations thus obtained are generally too thick and whitening, difficult to apply, and especially to spread, onto keratin materials, and give a sensation of heaviness and of difficulty in penetrating during application to the skin.

Thus, there is a need for cosmetic, dermatological or therapeutic compositions comprising C-glycoside derivatives, which may nevertheless be endowed with significant thickening, if need be.

There is also a need for cosmetic or therapeutic compositions whose viscosity is suitable for easy application to keratin materials and/or fibres and especially fluid compositions with a high particle content, which remain readily vaporizable.

The object of the present invention is, precisely, to satisfy these needs.

More specifically, the present invention relates to a cosmetic and/or pharmaceutical composition, especially a dermatological composition, comprising, in a physiologically acceptable medium containing an aqueous medium, at least one C-glycoside derivative and at least one associative polymer.

The inventors have observed, surprisingly, that the addition of a C-glycoside derivative to a composition comprising an associative polymer does not significantly affect the viscosity of the said composition and thus makes it possible to formulate it in a form that is suitable for handling it during its application.

What is more, this specific combination of C-glycoside-associative polymer also makes it possible to give a composition of the invention a pleasant appearance and, during its application, comfortable sensation properties.

According to yet another of its subjects, the present invention relates to a process for the non-therapeutic treatment or for making up keratin materials and/or fibres, comprising at least the step of applying to the said keratin materials and/or fibres at least one coat of a cosmetic composition in accordance with the invention.

Associative Polymer

Associative polymers are water-soluble polymers that are capable, in an aqueous medium, of reversibly combining with each other or with other molecules. Their chemical structure comprises hydrophilic zones and hydrophobic zones characterized by at least one fatty chain.

The associative polymers according to the invention may be of anionic, cationic, amphoteric or nonionic type.

a) Associative Polymers of Anionic Type

Among the associative polymers of anionic type, mention may be made of:

(I) copolymers comprising at least one hydrophilic unit and at least one fatty-chain allylic ether unit, more particularly those whose hydrophilic unit consists of an ethylenic unsaturated anionic monomer, more particularly still a vinylcarboxylic acid and most particularly an acrylic acid or a methacrylic acid or mixtures thereof, the fatty-chain allylic ether unit of which corresponds to the monomer of formula (I) below:
CH₂═C(R′)CH₂—O—Bₙ—R (I)
in which R′ denotes H or CH₃, B denotes an ethyleneoxy radical, n is zero or denotes an integer ranging from 1 to 100, R denotes a hydrocarbon-based radical chosen from alkyl, arylalkyl, aryl, alkylaryl and cycloalkyl radicals, containing from 8 to 30 carbon atoms, preferably 10 to 24 carbon atoms and even more particularly from 12 to 18 carbon atoms. A unit of formula (I) that is more particularly preferred is a unit in which R′ denotes H, n is equal to 10 and R denotes a stearyl (C₁₈) radical.

Mention may be made in particular of copolymers of methacrylic acid and of allylic ethers of C₈-C₃₀ fatty alcohols.

Anionic associative polymers of this type are described and prepared, according to an emulsion polymerization process, in patent EP-0 216 479.

Among these anionic associative polymers that are particularly preferred according to the invention are polymers formed from 20% to 60% by weight of acrylic acid and/or of methacrylic acid, from 5% to 60% by weight of lower alkyl(meth)acrylates, from 2% to 50% by weight of fatty-chain allylic ether of formula (I), and from 0% to 1% by weight of a crosslinking agent which is a well-known copolymerizable unsaturated polyethylenic monomer, for instance diallyl phthalate, allyl(meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), in particular those sold by the company Allied Colloids under the names Salcare SC 80® and Salcare SC 90®, which are aqueous 30% emulsions of a crosslinked terpolymer of methacrylic acid, of ethyl acrylate and of steareth-10 allylic ether (40/50/10).

(II) copolymers comprising at least one hydrophilic unit of unsaturated olefinic carboxylic acid type, and at least one hydrophobic unit of (C₁₀-C₃₀)alkyl ester of unsaturated carboxylic acid type.

Preferably, these polymers are chosen from those in which the hydrophilic unit of unsaturated olefinic carboxylic acid type corresponds to the monomer of formula (II) below: 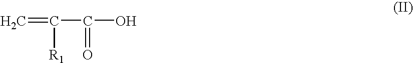
in which R₁ denotes H or CH₃ or C₂H₅, that is to say acrylic acid, methacrylic acid or ethacrylic acid units,
and in which the hydrophobic unit of (C₁₀-C₃₀)alkyl ester of unsaturated carboxylic acid type corresponds to the monomer of formula (III) below: 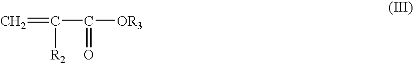
in which R₂ denotes H or CH₃ or C₂H₅ (that is to say acrylate, methacrylate or ethacrylate units) and preferably H (acrylate units) or CH₃ (methacrylate units), R₃ denoting a C₁₀-C₃₀ and preferably C₁₂-C₂₂ alkyl radical.

Mention may be made in particular of copolymers of unsaturated carboxylic acids and of unsaturated C₁₀-C₃₀ carboxylates.

(C₁₀-C₃₀) alkyl esters of unsaturated carboxylic acids according to the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. Nos. 3,915,921 and 4,509,949.

Among the anionic associative polymers of this type that will be used more particularly are polymers formed from a monomer mixture comprising:

(i) essentially acrylic acid,

(ii) an ester of formula (III) described above in which R₂ denotes H or CH₃, R₃ denoting an alkyl radical containing from 12 to 22 carbon atoms,

(iii) and a crosslinking agent, which is a well-known copolymerizable polyethylenic unsaturated monomer, for instance diallyl phthalate, allyl(meth)acrylate, divinylbenzene, (poly)ethylene glycol dimethacrylate and methylenebisacrylamide.

Among the anionic associative polymers of this type that will be used more particularly are those consisting of from 95% to 60% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among the said above polymers, those most particularly preferred according to the present invention are the products sold by the company Goodrich under the trade names Pemulen TR1®, Pemulen TR2® and Carbopol 1382®, and even more preferentially Pemulen TR1®, and the product sold by the company SEPPIC under the name Coatex SX®.

(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.

(IV) acrylic terpolymers obtained from (a) an α,β-ethylenically unsaturated carboxylic acid, (b) a non-surfactant α,β-ethylenically unsaturated monomer other than (a), and (c) a nonionic surfactant monomer obtained by reacting an ethylenically unsaturated monoisocyanate with a monohydric surfactant.

In particular, such polymers may be obtained from an α,β-ethylenically unsaturated carboxylic acid, and from a nonionic urethane monomer that is the product of reaction of a monohydric nonionic amphiphilic compound with a monoethylenically unsaturated isocyanate.

More particularly, such a polymer may comprise, relative to the total weight of the terpolymer:

(a) from about 20% to 70% by weight and preferably from 25% to 55% by weight of an α,β-ethylenically unsaturated carboxylic acid,

(b) from about 20% to 80% by weight and preferably from 30% to 65% by weight of a non-surfactant ethylenically unsaturated monomer other than (a), and

(c) from about 0.5% to 60% by weight and preferably from 10% to 50% by weight of a nonionic urethane monomer that is the product of reaction of a monohydric nonionic amphiphilic compound with a monoethylenically unsaturated isocyanate.

Such terpolymers are described in greater detail hereinbelow in the present description.

(V) copolymers comprising among their monomers a carboxylic acid containing α,β-monoethylenic unsaturation and an ester of a carboxylic acid containing α,β-monoethylenic unsaturation and of an oxyalkylenated fatty alcohol.

Preferentially, these compounds also comprise as monomer an ester of a carboxylic acid containing α,β-monoethylenic unsaturation and of a C₁-C₄ alcohol.

An example of a compound of this type that may be mentioned is Aculyn 22®sold by the company Rohm & Haas, which is a methacrylic acid/ethyl acrylate/stearyl methacrylate oxyalkylenated terpolymer, or Aculyn 28 (terpolymer of methacrylic acid/ethyl acrylate/oxyethylenated (25 EO) behenyl methacrylate).

b) Associative Polymers of Cationic Type

Among the associative polymers of cationic type that may be mentioned are:

(I) the cationic associative polyurethanes whose family has been described in patent application FR-A-2 811 993; it may be represented by the general formula (IV) below:
R—X—(P)ₙ-[L-(Y)ₘ]ᵣ-L′-(P′)ₚ—X′-R′ (IV)
in which:
R and R′, which may be identical or different, represent a hydrophobic group or a hydrogen atom;
X and X′, which may be identical or different, represent a group comprising an amine function optionally bearing a hydrophobic group, or alternatively a group L″;
L, L′ and L″, which may be identical or different, represent a group derived from a diisocyanate;
P and P′, which may be identical or different, represent a group comprising an amine function optionally bearing a hydrophobic group;
Y represents a hydrophilic group;
r is an integer between 1 and 100, preferably between 1 and 50 and in particular between 1 and 25;
n, m and p each range, independently of each other, from 0 to 1000; the molecule containing at least one protonated or quaternized amine function and at least one hydrophobic group.

In one preferred embodiment of these polyurethanes, the only hydrophobic groups are the groups R and R′ at the chain ends.

One preferred family of cationic associative polyurethanes is the one corresponding to formula (IV) described above and in which:
R and R′ both independently represent a hydrophobic group,
X and X′ each represent a group L″,
n and p are between 1 and 1000, and
L, L′, L″, P, P′, Y and m have the meaning given above.

Another preferred family of cationic associative polyurethanes is the one corresponding to formula (IV) above in which:
R and R′ both independently represent a hydrophobic group,
X and X′ each represent a group L″,
n and p are 0, and
L, L′, L″, Y and m have the meaning given above.

The fact that n and p are 0 means that these polymers do not comprise units derived from a monomer containing an amine function, incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide, a sulfate, etc.

Yet another preferred family of cationic associative polyurethanes is the one corresponding to formula (IV) above in which:

R and R′ both independently represent a hydrophobic group,

X and X′ both independently represent a group comprising a quaternary amine,

n and p are zero, and

L, L′, Y and m have the meaning given above.

The number-average molecular mass of the cationic associative polyurethanes is preferably between 400 and 500 000, in particular between 1000 and 400 000 and ideally between 1000 and 300 000.

The term “hydrophobic group” means a radical or polymer containing a saturated or unsaturated, linear or branched hydrocarbon-based chain, which may contain one or more heteroatoms such as P, O, N or S, or a radical containing a perfluoro or silicone chain.

When the hydrophobic group denotes a hydrocarbon-based radical, it comprises at least 10 carbon atoms, preferably from 10 to 30 carbon atoms, in particular from 12 to 30 carbon atoms and more preferably from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer such as, for example, polybutadiene.

When X and/or X′ denote(s) a group comprising a tertiary or quaternary amine, X and/or X′ may represent one of the following formulae: 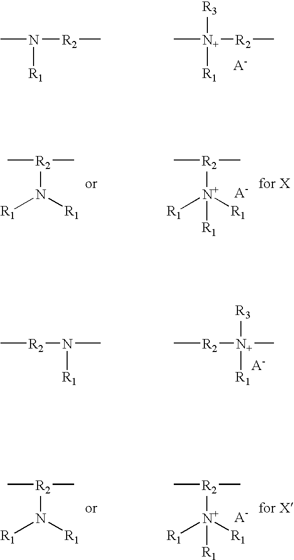
in which:
R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P;
R₁ and R₃, which may be identical or different, denote a linear or branched C₁-C₃₀ alkyl or alkenyl radical or an aryl radical, at least one of the carbon atoms possibly being replaced with a heteroatom chosen from N, S, O and P;
A⁻ is a physiologically acceptable counterion.

The groups L, L′ and L″ represent a group of formula: 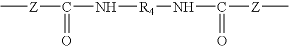
in which:
Z represents —O—, —S— or —NH—; and
R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P.

The groups P and P′ comprising an amine function may represent at least one of the following formulae: 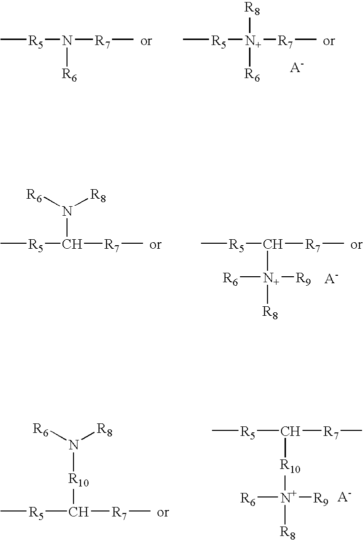
in which:
R₅ and R₇ have the same meanings as R₂ defined above;
R₆, R₈ and R₉ have the same meanings as R₁ and R₃ defined above;
R₁₀ represents a linear or branched, optionally unsaturated alkylene group possibly containing one or more heteroatoms chosen from N, O, S and P; and
A⁻ is a physiologically acceptable counterion.

As regards the meaning of Y, the term “hydrophilic group” means a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment of the invention, mention may be made, for example, of polyethers, sulfonated polyesters, sulfonated polyamides or a mixture of these polymers. The hydrophilic compound is preferentially a polyether and in particular a poly(ethylene oxide) or poly(propylene oxide).

The cationic associative polyurethanes of formula (IV) according to the invention are formed from diisocyanates and from various compounds with functions containing a labile hydrogen. The functions containing a labile hydrogen may be alcohol, primary or secondary amine or thiol functions, giving, after reaction with the diisocyanate functions, polyurethanes, polyureas and polythioureas, respectively. The term “polyurethanes” in the present invention encompasses these three types of polymer, namely polyurethanes per se, polyureas and polythioureas, and also copolymers thereof.

A first type of compound involved in the preparation of the polyurethane of formula (IV) is a compound comprising at least one unit containing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:
HZ-(P)ₙ-ZH
or
HZ-(P′)ₚ-ZH
in which Z, P, P′, n and p are as defined above.

Examples of compounds containing an amine function that may be mentioned include N-methyldiethanolamine, N-tert-butyldiethanolamine and N-sulfoethyldiethanolamine.

The second compound involved in the preparation of the polyurethane of formula (IV) is a diisocyanate corresponding to the formula:
O═C═N—R₄—N═C═O
in which R₄ is as defined above.

By way of example, mention may be made of methylenediphenyl diisocyanate, methylenecyclohexane diisocyanate, isophorone diisocyanate, toluene diisocyanate, naphthalene diisocyanate, butane diisocyanate and hexane diisocyanate.

A third compound involved in the preparation of the polyurethane of formula (IV) is a hydrophobic compound intended to form the terminal hydrophobic groups of the polymer of formula (IV).

This compound consists of a hydrophobic group and of a function containing a labile hydrogen, for example a hydroxyl, primary or secondary amine, or thiol function.

By way of example, this compound may be a fatty alcohol such as, in particular, stearyl alcohol, dodecyl alcohol or decyl alcohol. When this compound comprises a polymeric chain, it may be, for example, α-hydroxyl hydrogenated polybutadiene.

The hydrophobic group of the polyurethane of formula (IV) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and especially a poly(ethylene oxide) or poly(propylene oxide).

The hydrophilic group termed Y in formula (IV) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.

(II) quaternized cellulose derivatives and polyacrylates containing non-cyclic amine side groups.

The quaternized cellulose derivatives are, in particular,
quaternized celluloses modified with groups comprising at least one fatty chain, such as alkyl, arylalkyl or alkylaryl groups containing at least 8 carbon atoms, or mixtures thereof,
quaternized hydroxyethylcelluloses modified with groups comprising at least one fatty chain, such as alkyl, arylalkyl or alkylaryl groups containing at least 8 carbon atoms, or mixtures thereof.

The alkyl radicals borne by the above quaternized celluloses or hydroxyethylcelluloses preferably contain from 8 to 30 carbon atoms. The aryl radicals preferably denote phenyl, benzyl, naphthyl or anthryl groups.

Examples of quaternized alkylhydroxyethylcelluloses containing C₈-C₃₀ fatty chains that may be mentioned include the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Amerchol, and the products Crodacel QM®, Crodacel QL® (C₁₂ alkyl) and Crodacel QS® (C₁₈ alkyl) sold by the company Croda.

(III) the cationic polyvinyllactams whose family was described by the Applicant in patent application FR-0 101 106 and patent application WO 00/68282.

The said polymers comprise:

a) at least one monomer of vinyllactam or alkylvinyllactam type;

b) at least one monomer of structure (V) or (VI) below: 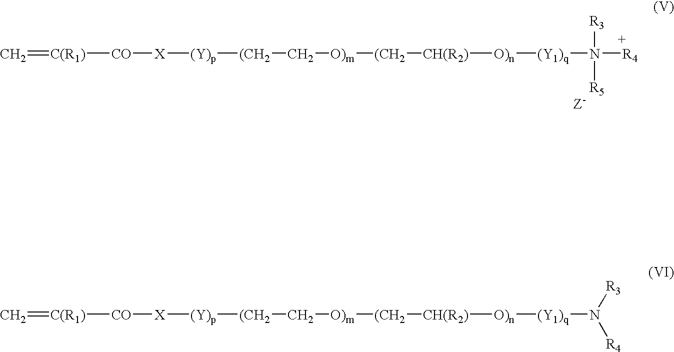
in which:
X denotes an oxygen atom or a radical NR₆,
R₁ and R₆ denote, independently of each other, a hydrogen atom or a linear or branched C₁-C₅ alkyl radical,
R₂ denotes a linear or branched C₁-C₄ alkyl radical,
R₃, R₄ and R₅ denote, independently of each other, a hydrogen atom, a linear or branched C₁-C₃₀ alkyl radical or a radical of formula (VII):
   —(Y₂)ᵣ—(CH₂—CH(R₇)—O)ₓ—R₈ (VII)
Y, Y₁ and Y₂ denote, independently of each other, a linear or branched C₂-C₁₆ alkylene radical,
R₇ denotes a hydrogen atom or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of each other, either the value 0 or the value 1,
m and n denote, independently of each other, an integer ranging from 0 to 100,
x denotes an integer ranging from 1 to 100,
Z denotes an organic or mineral acid anion,
   with the proviso that:
at least one of the substituents R₃, R₄, R₅ or R₈ denotes a linear or branched C₉-C₃₀ alkyl radical,
if m or n is other than zero, then q is equal to 1,
if m or n is equal to zero, then p or q is equal to 0.

The cationic poly(vinyllactam) polymers according to the invention may be crosslinked or non-crosslinked and may also be block polymers.

Preferably, the counterion Z⁻ of the monomers of formula (V) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferably, the monomer b) is a monomer of formula (V) for which, even more preferably, m and n are equal to 0.

The vinyllactam or alkylvinyllactam monomer is preferably a compound of structure (VIII): 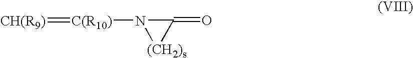
in which:
s denotes an integer ranging from 3 to 6,
R₉ denotes a hydrogen atom or a C₁-C₅ alkyl radical,
R₁₀ denotes a hydrogen atom or a C₁-C₅ alkyl radical,
   with the proviso that at least one of the radicals R₉ and R₁₀ denotes a hydrogen atom.

Even more preferably, the monomer (VIII) is vinylpyrrolidone.

The cationic poly(vinyllactam) polymers according to the invention may also contain one or more additional monomers, preferably cationic or nonionic monomers.

As compounds that are more particularly preferred according to the invention, mention may be made of the following terpolymers comprising at least:

a) one monomer of formula (VIII),

b) one monomer of formula (V) in which p=1, q=0, R₃ and R₄ denote, independently of each other, a hydrogen atom or a C₁-C₅ alkyl radical and R₅ denotes a C₉-C₂₄ alkyl radical, and

c) one monomer of formula (VI) in which R₃ and R₄ denote, independently of each other, a hydrogen atom or a C₁-C₅ alkyl radical.

Even more preferably, terpolymers comprising, by weight, 40% to 95% of monomer (a), 0.1% to 55% of monomer (c) and 0.25% to 50% of monomer (b) will be used.

Such polymers are described in patent application WO 00/68282, the content of which forms an integral part of the invention.

As cationic poly(vinyllactam) polymers according to the invention, vinylpyrrolidone/dimethylaminopropylmethacrylamide/dodecyldimethylmethacrylamido-propylammonium tosylate terpolymers, vinylpyrrolidone/dimethylaminopropylmethacryl-amide/cocoyldimethylmethacrylamidopropylammonium tosylate terpolymers, vinyl pyrrolidone/dimethylaminopropylmethacrylamide/lauryldimethylmethacrylamidopropyl-ammonium tosylate or chloride terpolymers are used in particular.

The weight-average molecular mass of the cationic poly(vinyllactam) polymers according to the present invention is preferably between 500 and 20 000 000. It is more particularly between 200 000 and 2 000 000 and even more preferably between 400 000 and 800 000.

The weight-average molecular mass of the cationic poly(vinyllactam) polymers according to the present invention is preferably between 1000 and 20 000 000. It is more particularly between 600 000 and 2 000 000 and even more preferentially between 500 000 and 900 000.

c) Associative Polymers of Amphoteric Type

The amphoteric associative polymers are preferably chosen from those comprising at least one non-cyclic cationic unit. Even more particularly, those prepared from or comprising I mol % to 20 mol %, preferably 1.5 mol % to 15 mol % and even more particularly 1.5 mol % to 6 mol %, relative to the total number of moles of monomers, of monomer comprising a fatty chain are preferred.

The amphoteric associative polymers that are preferred according to the invention comprise, or are prepared by copolymerizing:
1) at least one monomer of formula (IXa) or (IXb): 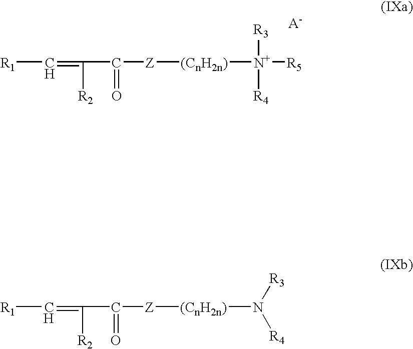
in which:
R₁ and R₂, which may be identical or different, represent a hydrogen atom or a methyl radical,
R₃, R₄ and R₅, which may be identical or different, represent a linear or branched alkyl radical containing from 1 to 30 carbon atoms,
Z represents an NH group or an oxygen atom,
n is an integer from 2 to 5,
A⁻ is an anion derived from an organic or mineral acid, such as a methosulfate anion or a halide such as chloride or bromide;
   2) at least one monomer of formula (X)
   R₆—CH═CR₇—COOH (X)
   in which:
R₆ and R₇, which may be identical or different, represent a hydrogen atom or a methyl radical; and
   3) at least one monomer of formula (XI):
   R₆—CH═CR₇—COXR₈ (XI)
   in which
R₆ and R₇, which may be identical or different, represent a hydrogen atom or a methyl radical, X denotes an oxygen or nitrogen atom and R₈ denotes a linear or branched alkyl radical containing from 1 to 30 carbon atoms;
   at least one of the monomers of formula (IXa), (IXb) or (XI) comprising at least one fatty chain.

The monomers of formulae (IXa) and (IXb) of the present invention are preferably chosen from the group consisting of:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate,
dimethylaminopropylmethacrylamide, dimethylaminopropylacrylamide,
   these monomers optionally being quaternized, for example with a C₁-C₄ alkyl halide or a C₁-C₄ dialkyl sulfate.

More particularly, the monomer of formula (IXa) is chosen from acrylamidopropyltrimethylammonium chloride and methacrylamidopropyltrimethyl-ammonium chloride.

The monomers of formula (X) of the present invention are preferably chosen from the group consisting of acrylic acid, methacrylic acid, crotonic acid and 2-methylcrotonic acid. More particularly, the monomer of formula (X) is acrylic acid.

The monomers of formula (XI) of the present invention are preferably chosen from the group consisting of C₁₂-C₂₂ and more particularly C₁₆-C₁₈ alkyl acrylates or methacrylates.

The monomers constituting the fatty-chain amphoteric polymers of the invention are preferably already neutralized and/or quaternized.

The ratio of the number of cationic charges/anionic charges is preferably equal to about 1.

The amphoteric associative polymers according to the invention preferably comprise from 1 mol % to 10 mol % of the monomer comprising a fatty chain and preferably from 1.5 mol % to 6 mol %.

The weight-average molecular weights of the amphoteric associative polymers according to the invention may range from 500 to 50 000 000 and are preferably between 10 000 and 5 000 000.

The amphoteric associative polymers according to the invention may also contain other monomers such as nonionic monomers and in particular such as C₁-C₄ alkyl acrylates or methacrylates.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

d) Associative Polymers of Nonionic Type

According to the invention, the associative polymers of nonionic type are preferably chosen from:

(1) hydroxypropyl guars modified with groups comprising at least one fatty chain, such as the product Esaflor HM 22® (C₂₂ alkyl chain) sold by the Company Lamberti, and the products RE210-18® (C₁₄ alkyl chain) and RE205-1® (C₂₀ alkyl chain) sold by the company Rhône-Poulenc.

(2) copolymers of vinylpyrrolidone and of fatty-chain hydrophobic monomers, examples of which that may be mentioned include:
the products Antaron V216® or Ganex V216® (vinylpyrrolidone/hexadecene copolymer) sold by the company I.S.P.
the products Antaron V220® or Ganex V220® (vinylpyrrolidone/eicosene copolymer) sold by the company I.S.P.
the products Antaron V660® or Ganex V660® (vinylpyrrolidone/1-triacontene (C30) copolymer sold by the company I.S.P.)

(3) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®.

(4) copolymers of hydrophilic methacrylates or acrylates and of hydrophobic monomers comprising at least one fatty chain, such as, for example, the polyethylene glycol methacrylate/lauryl methacrylate copolymer.

(5) polyurethane polyethers comprising in their chain both hydrophilic blocks usually of polyoxyethylenated nature and hydrophobic blocks which may be aliphatic sequences alone and/or cycloaliphatic and/or aromatic sequences.

(6) polymers with an aminoplast ether backbone containing at least one fatty chain, such as the Pure Thix® compounds sold by the company Sud-Chemie.

The nonionic associative polymers may also be chosen from copolymers of PEG-180, of tetramethoxymethylglycouril and of Laureth-50 or octoxynol-40.

Preferably, the polyurethane polyethers comprise at least two hydrocarbon-based lipophilic chains containing from 6 to 30 carbon atoms, separated by a hydrophilic block, the hydrocarbon-based chains possibly being pendent chains or chains at the end of a hydrophilic block. In particular, it is possible for one or more pendent chains to be included. In addition, the polymer may comprise a hydrocarbon-based chain at one end or at both ends of a hydrophilic block.

The polyurethane polyethers may be multiblock, in particular in triblock form. Hydrophobic blocks may be at each end of the chain (for example: triblock copolymer with a hydrophilic central block) or distributed both at the ends and in the chain (for example: multiblock copolymer). These same polymers may also be graft polymers or starburst polymers.

The nonionic fatty-chain polyurethane polyethers may be triblock copolymers in which the hydrophilic block is a polyoxyethylenated chain comprising from 50 to 1000 oxyethylene groups. The nonionic polyurethane polyethers comprise a urethane linkage between the hydrophilic blocks, whence arises the name.

By extension, also included among the nonionic fatty-chain polyurethane polyethers are those in which the hydrophilic blocks are linked to the lipophilic blocks via other chemical bonds.

As examples of fatty-chain nonionic polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂₋₁₄ alkyl chain, and the product Elfacos T212® containing a C₁₈ alkyl chain, from Akzo.

The product DW 1206B® from Rohm & Haas containing a C₂₀ alkyl chain and a urethane linkage, sold at a solids content of 20% in water, may also be used.

It is also possible to use solutions or dispersions of these polymers, especially in water or in aqueous-alcoholic medium. Examples of such polymers that may be mentioned are Rheolate® 255, Rheolate® 278 and Rheolate® 244 sold by the company Rheox. The products DW 1206F and DW 1206J sold by the company Rohm & Haas may also be used.

The polyurethane polyethers that may be used according to the invention are in particular those described in the article by G. Fonnum, J. Bakke and Fk. Hansen—Colloid Polym. Sci 271, 380.389 (1993).

Such polyurethane polyethers are especially sold by the company Rohm & Haas under the names Aculyn 46® and Aculyn 44®: Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexylisocyanate) (SMDI), at 35% by weight in a mixture of propylene glycol (39%) and water (26%).

According to one embodiment, a polymer that is suitable for use in the invention may be chosen from anionic associative polymers (for instance Viscophobe (DB1000) considered in the examples).

According to this particular embodiment, the associative polymer is more particularly an acrylic terpolymer obtained from (a) an α,β-ethylenically unsaturated carboxylic acid, (b) a non-surfactant ethylenically unsaturated monomer other than (a), and (c) a nonionic urethane monomer that is the product of reaction of a monohydric nonionic amphiphilic compound with a monoethylenically unsaturated isocyanate.

The acrylic terpolymer used in accordance with the invention is soluble or swellable in alkalis. It is preferably characterized in that it comprises, relative to the total weight of the terpolymer:

(a) about 20% to 70% by weight, preferably 25% to 55% by weight, of an α,β-monoethylenically unsaturated carboxylic acid;

(b) about 20% to 80% by weight, preferably 30% to 65% by weight, of a non-surfactant monoethylenically unsaturated monomer other than (a), and

(c) about 0.5% to 60% by weight, preferably 10% to 50% by weight, of a nonionic urethane monomer which is the product of reaction of a monohydric nonionic surfactant with a monoethylenically unsaturated monoisocyanate.

Such terpolymers are especially described in patent application EP-A-0 173 109.

The carboxylic acid containing α,β-monoethylenic unsaturation (a) can be chosen from many acids and in particular acrylic acid, methacrylic acid, itaconic acid and maleic acid. It is preferably methacrylic acid.

The terpolymer contains a monoethylenically unsaturated monomer (b) that has no surfactant properties. The preferred monomers are those which give polymers that are water-insoluble when they are homopolymerized and are illustrated by C₁-C₄ alkyl acrylates and methacrylates such as methyl acrylate, ethyl acrylate and butyl acrylate, or corresponding methacrylates. The monomers more particularly preferred are methyl acrylate and ethyl acrylate. Other monomers that may be used are styrene, vinyltoluene, vinyl acetate, acrylonitrile and vinylidene chloride. Non-reactive monomers are preferred, such monomers being those in which the single ethylenic group is the only group that is reactive under the polymerization conditions. However, monomers which contain groups that are reactive under the action of heat, for instance hydroxyethyl acrylate, may optionally be used.

The monohydric nonionic amphiphilic compounds used to obtain the nonionic urethane monomer (c) are well known and are generally alkoxylated hydrophobic compounds containing an alkylene oxide forming the hydrophilic part of the molecule. The hydrophobic compounds are generally constituted by an aliphatic alcohol or an alkylphenol in which a carbon-based chain containing at least six carbon atoms constitutes the hydrophobic part of the amphiphilic compound.

The preferred monohydric nonionic amphiphilic compounds are compounds having the formula (I) below:
R—(OCH₂CHR′)ₘ—(OCH₂CH₂)ₙ—OH (I)
in which R is chosen from alkyl groups containing from 6 to 30 carbon atoms and aralkyl groups having alkyl radicals containing from 8 to 30 carbon atoms, R′ is chosen from alkyl groups containing from 1 to 4 carbon atoms, n is an average number ranging from about 6 to 150 and m is an average number ranging from about 0 to 50, on condition that n is at least as large as m and that n+m=6 to 150.

In particular, in the compounds of formula (I), the group R is chosen from alkyl groups containing from 18 to 26 carbon atoms and (C₈-C₁₃)alkyl-phenyl groups; the group R′ is a methyl group; m=0 and n=6 to 150. The compound of formula (I) may in particular be an oxyalkylene derivative and in particular an oxyethylene derivative of an aliphatic alcohol of plant origin and especially of behenyl alcohol, the radical R in formula (I) then being the behenyl radical.

The monoethylenically unsaturated isocyanate used to form the nonionic urethane monomer (c) may be chosen from very varied compounds. A compound containing any copolymerizable unsaturation such as acrylic, methacrylic or allylic unsaturation may be used. The preferred monoethylenically unsaturated isocyanate is α,α-dimethyl-m-isopropenyl benzyl isocyanate.

The acrylic terpolymer defined above is obtained by copolymerization in aqueous dispersion of the components (a), (b) and (c), this copolymerization being entirely trivial and described especially in document EP-A-0 173 109.

As terpolymers that may be used according to the invention, mention may be made of the product of reaction of methacrylic acid as component (a), of ethyl acrylate as component (b) and of a nonionic urethane macromonomer as component (c), having the structure (II) below: 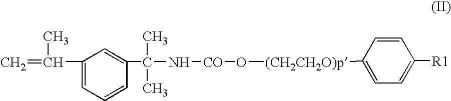
in which p′ ranges from 6 to 150 and is preferably equal to 30 and R1 is chosen from alkyl radicals containing from 8 to 13 carbon atoms, as described in Example 3 of document EP-A-0 173 109.

The preferred acrylic terpolymer used according to the invention is obtained from methacrylic acid as component (a), methyl acrylate as component (b) and a nonionic urethane macromonomer as component (c), having the structure (III) below: 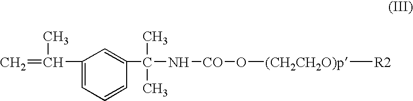
in which p ranges from 6 to 150 and R2 is chosen from linear alkyl radicals containing from 18 to 26 and preferably from 20 to 24 carbon atoms. Preferably, the radical R2 in the compound of formula (III) is a radical of plant origin, such as the behenyl radical.

The terpolymers used according to the invention are generally in aqueous dispersion.

Terpolymers that are most particularly suitable for use in the invention are maleic anhydride/C₃₀-C₃₈ α-olefin/alkyl maleate terpolymers, the methacrylic acid/ethyl acrylate/ethyl acrylate/oxyethylenated (25 EO) behenyl methacrylate terpolymer and in particular the methacrylic acid/methyl acrylate/ethoxylated (40 EO) behenyl dimethyl-meta-isopropenyl benzyl isocyanate terpolymer as an aqueous dispersion at 25% by weight, sold under the name Viscophobe DB 1000® by the company Amerchol.

According to one embodiment, a composition of the invention comprises at least from about 0.01% to 30% by weight, in particular from about 0.05% to 15% by weight, preferably from about 0.1% to 10% by weight and more particularly from about 0.1% to 5% by weight of associative polymer(s) relative to the total weight of the composition.

C-Glycoside Derivatives

A C-glycoside derivative that is suitable for use in the invention may be a compound of general formula (I) below: 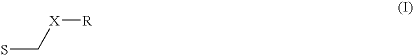
in which:

R represents:
a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical;
a saturated C₁-C₂₀ and in particular C₁-C₁₀ or unsaturated C₂-C₂₀ and in particular C₂-C₁₀, or saturated or unsaturated, branched or cyclic C₃-C₂₀ and in particular C₃-C₁₀ linear hydrofluoroalkyl or perfluoroalkyl radical;
   the hydrocarbon-based chain constituting the said radicals possibly being, where appropriate, interrupted with 1, 2, 3 or more heteroatoms chosen from:
an oxygen,
a sulfur,
a nitrogen, and
a silicon,
   and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
   with R₄ and R₅ possibly representing, independently of each other, a hydrogen atom or a saturated C₁-C₃₀ and in particular C₁-C₁₂ or unsaturated C₂-C₃₀ and in particular C₂-C₁₂, or a saturated or unsaturated, branched or cyclic C₃-C₃₀ and in particular C₃-C₁₂ alkyl, perfluoroalkyl or hydrofluoroalkyl radical; or a C₆-C₁₀ aryl radical

X represents a radical chosen from the groups: 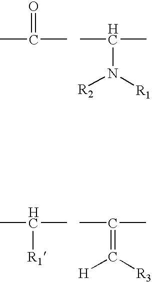
with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;

S represents a monosaccharide or a polysaccharide comprising up to 20 sugar units and in particular up to 6 sugar units, in pyranose and/or furanose form and of L and/or D series, the said mono- or polysaccharide possibly being substituted with a mandatory free hydroxyl group, and optionally one or more optionally protected amine function(s), and

the bond S—CH₂—X represents a bond of C-anomeric nature, which may be α or β,

and also the cosmetically acceptable salts thereof, the solvates thereof such as hydrates, and the isomers thereof.

In the context of the present invention, the term “halogen” means chlorine, fluorine, bromine or iodine.

The term “aryl” denotes an aromatic ring such as phenyl, optionally substituted with one or more C₁-C₄ alkyl radicals.

The term “C₃-C₈ cycloalkyl” denotes an aliphatic ring containing from 3 to 8 carbon atoms, for example including cyclopropyl, cyclopentyl and cyclohexyl.

Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, it is possible to use a C-glycoside derivative corresponding to formula (I) for which S may represent a monosaccharide or a polysaccharide containing up to 6 sugar units, in pyranose and/or furanose form and of L and/or D series, the said monosaccharide or polysaccharide containing at least one hydroxyl function that is mandatorily free and/or optionally one or more amine functions that are mandatorily protected, X and R otherwise retaining all the definitions given above.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine, and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, an oligosaccharide containing at least one xylose advantageously chosen from xylobiose, methyl-β-xylobioside, xylotriose, xylotetraose, xylopentaose and xylohexaose and especially xylobiose, which is composed of two xylose molecules linked via a 1-4 bond.

More particularly, S may represent a monosaccharide chosen from D-glucose, D-xylose, L-fucose, D-galactose and D-maltose, especially D-xylose.

According to another embodiment of the invention, it is possible to use C-glycoside derivatives corresponding to formula (I) for which X represents a group chosen from —CO—, —CH(OH)—, —CH(NR₁R₂)— and —CH(R)—, in particular —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferentially a —CH(OH)— group, S and R otherwise conserving all of the definitions given above.

According to another embodiment of the invention, it is possible to use a C-glycoside derivative corresponding to formula (I) for which R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical; and optionally substituted as described above, S and X otherwise conserving all the definitions given above. Preferably, R denotes a linear C₁-C₄ and especially C₁-C₃ radical optionally substituted with —OH, —COOH or —COOR″₂, R″₂ being a saturated C₁-C₄ alkyl radical, especially ethyl.

Preferentially, R denotes an unsubstituted linear C₁-C₄ and especially C₁-C₂ alkyl radical, in particular ethyl.

Among the C-glycoside derivatives of formula (I) that are preferably used are those for which:
R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical, optionally substituted as described above;
S represents a monosaccharide as described above;
X represents —CO—, —CH(OH)—, —CH(NR₁R₂)— or —CH(R)—, as defined above.

Preferably, a C-glycoside derivative of formula (I) is used, for which:
R denotes a linear C₁-C₄ and especially C₁-C₃ radical, optionally substituted with —OH, —COOH or —COOR″₂, R″₂ being a saturated C₁-C₄ alkyl radical, especially ethyl;
S represents a monosaccharide as described above;
   X represents a group chosen from —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferentially a —CH(OH)— group.

Preferentially, a C-glycoside derivative of formula (I) is used, for which:
R denotes an unsubstituted linear C₁-C₄ and especially C₁-C₂ alkyl radical, in particular ethyl;
S represents a monosaccharide as described above; especially D-glucose, D-xylose, N-acetyl-D-glucosamine or L-fucose, in particular D-xylose;
X represents a group chosen from —CO—, —CH(OH)— and —CH(NH₂)— and preferentially a —CH(OH)— group.

The salts that are acceptable for the non-therapeutic use of the compounds described in the present invention comprise conventional non-toxic salts of the said compounds such as those formed from organic or inorganic acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic groups. They may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

When the compound of formula (I) comprises an acid group, neutralization of the acid group(s) may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine, dimethylamino-2-propanol or 2-amino-2-(hydroxymethyl)-1,3-propanediol. Mention may also be made of lysine or 3-(dimethylamino)propylamine.

The solvates that are acceptable for the compounds described in the present invention comprise conventional solvates such as those formed during the final step of preparation of the said compounds due to the presence of solvents. Examples that may be mentioned include the solvates due to the presence of water or of linear or branched alcohols, for instance ethanol or isopropanol.

Among the C-glycoside derivatives of formula (I) used according to the invention, the ones that are most particularly considered are:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl 3-methyl-4-(C-β-D-xylopyranoside)butyrate;
12. ethyl 3-methyl-4-(C-α-D-xylopyranoside)butyrate;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl 3-methyl-4-(C-β-D-fucopyranoside)butyrate;
50. ethyl 3-methyl-4-(C-α-D-fucopyranoside)butyrate;
51. ethyl 3-methyl-4-(C-β-L-fucopyranoside)butyrate;
52. ethyl 3-methyl-4-(C-α-L-fucopyranoside)butyrate;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)-4-ketopentanoic acid;
72. 5-(C-α-L-fucopyranoside)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl 3-methyl-4-(C-β-D-glucopyranosyl)butyrate;
92. ethyl 3-methyl-4-(C-α-D-glucopyranosyl)butyrate;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylaminohexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl 3-methyl-4-(β-D-galactopyranosyl)butyrate;
120. ethyl 3-methyl-4-(α-D-galactopyranosyl)butyrate;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propane-2′-one;
146. 3′-(acetamido-C-α-D-glucopyranosyl)propane-2′-one;
147. 1-(acetamido-C-β-D-glucopyranosyl)-2-hydroxylpropane;
148. 1-(acetamido-C-β-D-glucopyranosyl)-2-aminopropane;
149. 1-(acetamido-C-β-D-glucopyranosyl)-2-phenylaminopropane;
150. 1-(acetamido-C-α-D-glucopyranosyl)-2-phenylaminopropane;
151. ethyl 3-methyl-4-(acetamido-C-β-D-glucopyranosyl)butyrate;
152. ethyl 3-methyl-4-(acetamido-C-α-D-glucopyranosyl)butyrate;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl)hexane-2,6-diol;
162. 1-(acetamido-C-β-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl)pentane-2,5-diol;
172. 1-(acetamido-C-α-D-glucopyranosyl)pentane-2,5-diol.

As non-limiting illustrations of C-glycoside derivatives that are more particularly suitable for use in the invention, mention may be made especially of the following derivatives:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1 (C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1 (C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxypropane, and better still C-β-D-xylopyranoside-2-hydroxypropane, may advantageously be used for the preparation of a composition according to the invention.

According to one embodiment, C-β-D-xylopyranoside-2-hydroxypropane may be used in the form of a solution containing 30% by weight of active agent in a 60/40 water/1,2-propanediol mixture, such as the product sold under the name Mexoryl SBB® by the company Chimex.

Needless to say, according to the invention, a C-glycoside derivative corresponding to formula (I) may be used alone or as a mixture with other C-glycoside derivatives and in all proportions.

A C-glycoside derivative that is suitable for use in the invention may especially be obtained via the synthetic method described in document WO 02/051 828.

The amount of C-glycoside derivative to be used in a composition according to the invention depends on the desired cosmetic or therapeutic effect, and may thus vary within a wide range.

A person skilled in the art can readily determine the appropriate amounts, on the basis of his general knowledge.

A composition in accordance with the invention may comprise a C-glycoside derivative in a proportion of about from 0.0001% to about 25% by weight relative to the total weight of the composition, in particular from about 0.001% to about 10% by weight and even more particularly between 0.05% and 5% by weight of C-glycoside derivative active material relative to the total weight of the composition.

According to one embodiment, a composition according to the invention may comprise at least one C-glycoside derivative and at least one associative polymer in particular of anionic type and especially of acrylic terpolymer type and more particularly as defined above, in a weight ratio ranging from about 0.002 to about 50 and more particularly from about 0.01 to about 15.

Protocol for Measuring the Viscosity

The viscosity of a composition of the invention may be measured according to any process known to those skilled in the art, and especially according to the following conventional process. Thus, the measurement may be performed at 25° C. using a Contraves TV or Rheomat 180 viscometer, equipped with a spindle rotating at 200 rpm. A person skilled in the art can select the spindle for measuring the viscosity from the spindles M1 and M2, on the basis of his general knowledge, so as to be able to perform the measurement.

The addition of a C-glycoside as defined above to an aqueous solution comprising an associative polymer according to the invention is reflected by a viscosity change of less than 20%, in particular less than or equal to 15% and in particular less than or equal to 10% measured relative to the viscosity of a solution comprising only the polymer.

A composition in accordance with the invention comprises a physiologically acceptable medium.

The term “physiologically acceptable medium” is intended to denote a medium that is compatible with human keratin materials and/or fibres, for instance, in a non-limiting manner, the skin, mucous membranes, the nails, the scalp and/or the hair.

This physiologically acceptable medium may at least comprise an aqueous phase optionally as a mixture with one or more organic solvents such as a C₁-C₈ alcohol, especially ethanol, isopropanol, tert-butanol, n-butanol, polyols, for instance glycerol, propylene glycol or butylene glycol, and polyol ethers.

A composition according to the invention may also comprise a fatty phase, which may comprise oils, gums or waxes usually used in the field of application under consideration.

Thus, according to one embodiment, a composition according to the invention may also comprise at least one fatty phase chosen from a fatty phase that is solid at room temperature (20-25° C.) and atmospheric pressure, and/or a fatty phase that is liquid at room temperature (20-25° C.) and atmospheric pressure.

A liquid fatty phase that is suitable for use in the invention may comprise a volatile oil, a non-volatile oil, and a mixture thereof. A volatile or non-volatile oil may be a hydrocarbon-based oil, especially of animal or plant origin, a synthetic oil, a silicone oil, a fluoro oil, or a mixture thereof.

A solid fatty phase that is suitable for use in the invention may be chosen, for example, from pasty fatty substances and gums, and mixtures thereof.

As oils or waxes that may be used in the invention, mention may be made of mineral oils (liquid petroleum jelly), plant oils (liquid fraction of shea butter, sunflower oil), animal oils (perhydrosqualene), synthetic oils (parceling oil), silicone oils or waxes (cyclomethicone) and fluoro oils (perfluoropolyethers), and beeswax, carnauba wax or paraffin wax. Fatty alcohols and fatty acids (stearic acid) may be added to these oils.

When a composition is an emulsion, the proportion of the fatty phase may range from 5% to 80% by weight and preferably from 5% to 50% by weight relative to the tot al weight of the composition. The oils, waxes, emulsifiers and coemulsifiers, other than the emulsifying polymers used in the present patent application, which may be used in the composition in emulsion form are chosen from those conventionally used in cosmetics.

An emulsifier and a coemulsifier may be present in a composition of the invention in a proportion ranging from 0.3% to 30% by weight and in particular from 0.5% to 20% by weight relative to the total weight of the composition.

An emulsion according to the invention may also contain lipid vesicles.

When a composition according to the invention is an oily solution or gel, the fatty phase may represent more than 90% of the total weight of the composition.

A composition according to the invention may also contain adjuvants that are common in the field under consideration, such as other surfactants or emulsifiers, hydrophilic or lipophilic gelling agents, hydrophilic or lipophilic additives, preserving agents, antioxidants, solvents, fragrances, fillers, UVA and/or UVB screening agents (soluble or insoluble, organic or mineral), pigments, fibres, chelating agents, odour absorbers, dyestuffs, and other cosmetic or pharmaceutical active agents.

The amounts of these various adjuvants are those conventionally used in cosmetics, and may be, for example, from 0.01% to 30% of the total weight of the composition. In general, the amounts are adjusted as a function of the formulation prepared. Depending on their nature, these adjuvants may be introduced into the fatty phase, into the aqueous phase and/or into lipid spherules.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and as lipophilic gelling agents that may be used, mention may be made of modified clays, for instance bentones, metal salts of fatty acids, for instance aluminium stearates, hydrophobic silica and polyethylene.

A composition of the invention may be in any conceivable galenical form.

In particular, a composition according to the invention may have the form of an aqueous, alcoholic or aqueous-alcoholic solution; a dispersion of the lotion or serum type; a water-in-oil, oil-in-water or multiple emulsion; a suspension; microcapsules or microparticles; vesicular dispersions of ionic and/or nonionic type; an aqueous or oily lotion or a lotion in serum form; capsules, granules, syrups or tablets; a mousse or a solid preparation; an aerosol composition also comprising a pressurized propellant.

A composition according to the invention may be in the form of a haircare composition, especially a shampoo, a hairsetting lotion, a medicated lotion, a styling cream or gel, a dye composition, especially for oxidation dyeing, restructuring lotions for the hair, a permanent-waving composition (especially a composition for the first stage of a permanent-waving operation), a lotion or gel for preventing hair loss, or an antiparasitic shampoo.

It may also be in the form of a cleansing, protective, treating or care composition for the face, the hands, the feet, the major anatomical folds or the body (for example a day cream, a night cream, a makeup-removing cream, an anti-sun composition, a protective or care body milk, an after-sun milk, a skincare lotion, gel or mousse, for example a cleansing lotion, or an artificial tanning composition); a facial or body makeup composition such as a foundation; a bath composition; a deodorizing composition comprising, for example, a bactericidal agent; an aftershave composition, a hair-removing composition; an insect-repelling composition; a pain-relief composition; a composition for treating certain skin diseases, for instance eczema, rosacea, psoriasis, lichens and severe pruritus.

When a composition according to the invention is intended for a use of peeling type, it may also be in any galenical form mentioned above, provided that it can be removed easily by rinsing, especially in the form of an aqueous gel or an aqueous or aqueous-alcoholic solution.

A composition according to the invention may be applied by any means that allows uniform distribution, and especially using cotton wool, a cotton tip, a brush, a gauze, a spatula or a pad, or alternatively by spraying, and may be removed by rinsing with water or using a mild detergent.

A composition according to the invention may be in a fluid form of vaporizable or non-vaporizable liquid type, or in the form of a paste, a direct or inverse emulsion, a gel or an impregnated support.

In particular, a composition according to the invention may be in a solid form, especially a compact, pulverulent or cast form, or in the form of a stick.

A composition according to the invention may also be in the form of a care product, an antisun or after-sun product, a daily photoprotective care product, a body product, a foundation to be applied to the face or the neck, a concealer product, a complexion corrector, a tinted cream, a makeup base for facial makeup or a body makeup composition.

A composition according to the invention may also comprise one or more additional cosmetic or therapeutic active agent(s).

Thus, a composition according to the present invention may in particular comprise at least:
one anti-ageing and/or anti-wrinkle agent,
one anti-glycation agent,
one NO-synthase inhibitor,
one agent acting on dermal or epidermal macromolecules and/or preventing their degradation,
one agent for modulating fibroblast or keratinocyte proliferation and/or keratinocyte differentiation, and
a muscle relaxant,
a depigmenting or pro-pigmenting agent,
an antimicrobial agent,
a tensioning agent,
an anti-pollution agent or free-radical scavenger,
a calmative,
a lipolytic active agent or an agent that has direct or indirect favourable activity on reducing adipose tissue,
an agent acting on the capillary circulation,
an antiacne agent, and
a moisturizer.

A composition according to the invention may be used for the purposes of improving the general condition of an epidermis, in particular the skin, and especially for maintaining or restoring its physiological functions and/or its aesthetic appearance.

Thus, a composition according to the invention may be advantageously used for combating ageing of the epidermis, for maintaining and/or stimulating moisturization and/or for combating skin dryness, for improving the tonicity of the skin, for maintaining or restoring the suppleness and elasticity of the skin, for improving the mineralization of the epidermis, for improving the vitality of the epidermis, for facilitating intercellular exchanges, and for combating chapping and the cracked appearance of the skin.

A composition according to the invention may be intended for cosmetic and/or dermatological use.

Other characteristics and advantages of the invention will emerge more clearly from the examples that follow, which are given as non-limiting illustrations. In the text hereinabove and hereinbelow, the proportions are given as weight percentages, unless otherwise mentioned.

### EXAMPLES

In the examples given below, the C-glycoside derivative is C-β-D-xylopyranoside-2-hydroxypropane. It is more particularly an aqueous solution containing 30% by weight of active material (AM) in a 60/40 water/propylene glycol mixture (sold under the name Mexoryl SBB® by the company Chimex).

### Examples 1 to 3

Three aqueous gels were prepared with three different thickening polymers (associative polymer according to the invention and two non-associative polymers outside the invention) and, for each of the polymers, the gel was prepared with or in the absence of C-β-D-xylopyranoside-2-hydroxypropane. The viscosity of the aqueous gels obtained was then measured after 24 hours of storage at room temperature (viscosity measured at 25° C. using a Contraves TV viscometer with an M3 spindle, after 10 minutes of rotation at 200 rpm).

| | | Example 1B |
|---|---|---|
| | Example 1A | (invention) |
| C-β-D-xylopyranoside-2-hydroxypropane as | 0 | 2.98%, |
| a solution at 30% by weight in a 60/40 | | i.e. 1% AM |
| water/propylene glycol mixture | | |
| Methacrylic acid/methyl acrylate/ | 2% | 2% |
| ethoxylated (40 EO) behenyl dimethyl-meta- | | |
| isopropenyl benzyl isocyanate terpolymer | | |
| (Viscophobe DB 1000 from Amerchol) | | |
| Sodium hydroxide | 1.5% | 1.5% |
| Water | qs 100% | qs 100% |
| Viscosity (mPa · s) | 5.1 | 4.4 |

| | | Example 2B |
|---|---|---|
| | Example 2A | BLANK |
| C-β-D-xylopyranoside-2-hydroxypropane as | 0 | 2.98%, |
| a solution at 30% by weight in a 60/40 | | i.e. 1% AM |
| water/propylene glycol mixture | | |
| Acrylamide/sodium acrylamido-2- | 3% | 3% |
| methylpropanesulfonate copolymer as an | | |
| inverse emulsion at 40% in isoparaffin/water | | |
| (Sepigel 305 from SEPPIC) | | |
| Water | qs 100% | qs 100% |
| Viscosity (mPa · s) | 3.16 | 2.07 |

| | | Example 3B |
|---|---|---|
| | Example 3A | BLANK |
| C-β-D-xylopyranoside-2-hydroxypropane as | 0 | 2.98%, |
| a solution at 30% by weight in a 60/40 | | i.e. 1% AM |
| water/propylene glycol mixture | | |
| Carboxyvinyl polymer (Carbopol 981 from | 3% | 3% |
| Noveon) | | |
| Water | qs 100% | qs 100% |
| Viscosity (mPa · s) | 9.5 | 7.2 |

These tests show that only the associative polymer Viscophobe DB 1000 in the presence of C-β-D-xylopyranoside-2-hydroxypropane shows a low variation in viscosity. Thus, the presence of the associative polymer makes it possible to maintain the viscosity of the aqueous gel in the presence of C-β-D-xylopyranoside-2-hydroxypropane.

### Example 4

### Fluid Containing Sugar Esters

| Compounds | % |
|---|---|
| Methylglucose sesquistearate (Tegocare PS from Goldschmidt) | 2 |
| Mixture of stearyl alcohol and cetylstearyl alcohol | 2 |
| (Promulgen G from Noveon) | |
| Cyclohexadimethylsiloxane | 10 |
| Shorea robusta (Beurre de Sale from Stearineries Dubois) | 6 |
| Apricot kernel oil | 2 |
| Oxyethylenated methylglucose sesquistearate (glucamate | 2 |
| SSE 20 from Noveon) | |
| Methacrylic acid/methyl acrylate/ethoxylated (40 EO) behenyl | 1 |
| dimethyl-meta-isopropenyl benzyl isocyanate terpolymer | |
| (Viscophobe DB 1000 from Amerchol) | |
| Preserving agent | 0.5 |
| Water | qs 100 |
| C-β-D-xylopyranoside-2-hydroxypropane as a solution at 30% | 1% AM |
| by weight in a 60/40 water/propylene glycol mixture | |

### Example 5

### Anti-Ageing Cream

| Compounds | % |
|---|---|
| Glyceryl mono/distearate/polyethylene glycol (100 EO) | 2.5 |
| stearate mixture (Arlacel 135 from Uniqema) | |
| Myrj 53 | 2.5 |
| Cetyl alcohol | 1 |
| Stearyl alcohol | 1 |
| Hydrogenated isoparaffin (6-8 mol of isobutylene) | 5 |
| (Parleam from NOF Corporation) | |
| Cyclopentasiloxane | 15 |
| Water | qs 100 |
| Preserving agent | 0.5 |
| Methacrylic acid/methyl acrylate/ethoxylated behenyl | 0.3 |
| dimethyl-meta-isopropenyl benzyl isocyanate terpolymer | |
| (Viscophobe DB 1000 from Amerchol) | |
| C-β-D-xylopyranoside-2-hydroxypropane as a solution at | 5% AM |
| 30% by weight in a 60/40 water/propylene glycol mixture | |

### Example 6

### Vaporizable Fluid Antisun Formula

| Compounds | Ex. A | Ex. B |
|---|---|---|
| Sequestrant | qs | qs |
| Triethanolamine | qs | qs |
| Butylmethoxydibenzoylmethane 4-tert-butyl-4′- | 1 | 3 |
| methoxydibenzoyl-methane (Parsol 1789 from | | |
| DSM Nutritional Products) | | |
| 2-Ethylhexyl salicylate (Neo Heliopan OS from | 0 | 5 |
| Symrise) | | |
| Ethylhexyl triazone (Uvinul T 150 from BASF) | 0.5 | 0.5 |
| Terephthalylidenedicamphorsulfonic acid | 1.5 | 1.5 |
| (Mexoryl SX from Chimex) | | |
| Octocrylene (Uvinul N 539 from BASF) | 10 | 10 |
| Titanium dioxide (MT 100 AQ from Tayca) | 0 | 3.5 |
| Diglycol cyclohexanedimethanol isophthalates | 2 | 2 |
| sulfoisophthalates copolymer (Eastman AQ 38S | | |
| Polymer from Eastman Chemical) | | |
| Methacrylic acid/methyl acrylate/ethoxylated behenyl | 0.5 | 0.5 |
| dimethyl-meta-isopropenyl benzyl isocyanate | | |
| terpolymer (Viscophobe DB 1000 from Amerchol) | | |
| Cyclohexasiloxane | 5 | 5 |
| Water | qs 100 | qs 100 |
| C₁₂-C₁₅ Alkyl benzoate (Finsolv TN from Finetex) | 6 | 1 |
| Glycerol | 3 | 3 |
| C-β-D-xylopyranoside-2-hydroxypropane as a | 3% AM | 3% AM |
| solution at 30% by weight in a 60/40 water/propylene | | |
| glycol mixture (Mexoryl SBB) | | |
| Preserving agent | qs | qs |

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic and/or pharmaceutical composition, especially a dermatological composition, comprising, in a physiologically acceptable medium containing an aqueous medium, at least one C-glycoside derivative and at least one associative polymer.

2. Composition according to the claim 1, in which the C-glycoside derivative corresponds to the general formula (I) below: 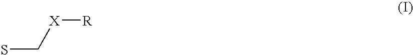
in which:
R represents:
a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical;
a saturated C₁-C₂₀ and in particular C₁-C₁₀ or unsaturated C₂-C₂₀ and in. particular C₂-C₁₀, or saturated or unsaturated, branched or cyclic C₃-C₂₀ and in particular C₃-C₁₀ linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
with R₄ and R₅ possibly representing, independently of each other, a hydrogen atom or a saturated C₁-C₃₀ and in particular C₁-C₁₂ or unsaturated C₂-C₃₀ and in particular C₂-C₁₂, or a saturated or unsaturated, branched or cyclic C₃-C₃₀ and in particular C₃-C₁₂ alkyl, perfluoroalkyl or hydrofluoroalkyl radical; or a C₆-C₁₀ aryl radical,
X represents a radical chosen from the groups: 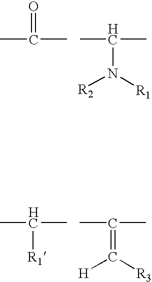
with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;
S represents a monosaccharide or a polysaccharide comprising up to 20 sugar units and in particular up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X represents a bond of C-anomeric nature, which may be a or β, and also the cosmetically acceptable salts thereof, the solvates thereof such as hydrates, and the isomers thereof.

3. Composition according to the claim 2, in which S represents a monosaccharide chosen from D-glucose, D-xylose, L-fucose, D-galactose and D-maltose, especially D-xylose.

4. Composition according to claim 2, in which X represents a group chosen from —CO—, —CH(OH)— and —CH(NH₂)—, and preferentially a —CH(OH)— group.

5. Composition according to claim 2, in which R denotes a linear C₁-C₄ and especially C₁-C₃ radical, optionally substituted with —OH, —COOH or —COOR″₂, R″₂ being a saturated C₁-C₄ alkyl radical, especially ethyl.

6. Composition according to claim 1, in which the C-glycoside derivative is chosen from:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

7. Composition according to claim 1, in which the C-glycoside derivative is chosen from C-β-D-xylopyranoside-2-hydroxypropane and C-α-D-xylopyranoside-2-hydroxypropane, and is more particularly C-β-D-xylopyranoside-2-hydroxypropane.

8. Composition according to claim 1, comprising from about 0.0001% to about 25% by weight relative to the total weight of the composition, of C-glycoside derivative active material relative to the total weight of the composition.

9. Composition according to claim 1, comprising from about 0.001% to about 10% by weight relative to the total weight of the composition, of C-glycoside derivative active material relative to the total weight of the composition.

10. Composition according to claim 1, in which the associative polymers are chosen from anionic, nonionic, amphoteric and cationic associative polymers.

11. Composition according to claim 1, comprising at least one anionic associative polymer chosen from copolymers of (meth)acrylic acid and of allylic ethers of C₈-C₃₀ fatty alcohols, copolymers of unsaturated carboxylic acids and of C₁₀-C₃₀ alkyl unsaturated carboxylates, terpolymers of maleic anhydride/C₃₀-C₃₈ α-olefin/alkyl maleate, acrylic terpolymers obtained from (a) an α,β-ethylenically unsaturated carboxylic acid, (b) a non-surfactant α,β-ethylenically unsaturated monomer other than (a), and (c) a nonionic surfactant monomer obtained by reacting an ethylenically unsaturated monoisocyanate and a monohydric surfactant, and copolymers of α,β-monoethylenically unsaturated carboxylic acids, of α,β-monoethylenically unsaturated carboxylic acid ester and of oxyalkylenated fatty alcohol.

12. Composition according to the claim 11, in which the anionic associative polymer is an acrylic terpolymer of an α,β-ethylenically unsaturated carboxylic acid, and of a nonionic urethane monomer that is the product of reaction of a monohydric nonionic amphiphilic compound with a monoethylenically unsaturated isocyanate.

13. Composition according to the claim 12, in which the acrylic terpolymer comprises, relative to the total weight of the terpolymer:
(a) from about 20% to 70% by weight of an α, β,-ethylenically unsaturated carboxylic acid,
(b) from about 20% to 80% by weight of a non-surfactant ethylenically unsaturated monomer other than (a), and
(c) from about 0.5% to 60% by weight of a nonionic urethane monomer that is the product of reaction of a monohydric nonionic amphiphilic compound with a monoethylenically unsaturated isocyanate.

14. Composition according to claim 11, wherein the associative polymer is a terpolymer, and in particular the methacrylic acid/methyl acrylate/ethoxylated behenyl dimethyl-meta-isopropenyl benzyl isocyanate terpolymer.

15. Composition according to claim 11, in which the nonionic associative polymers are chosen from hydroxypropyl guars modified with groups comprising at least one fatty chain, copolymers of vinylpyrrolidone and of fatty-chain hydrophobic monomers, copolymers of C₁-C₆ alkyl(meth)acrylates and of amphiphilic monomers comprising at least one fatty chain, copolymers of hydrophilic (meth)acrylates and of hydrophobic monomers comprising at least one fatty chain, polyether-polyurethanes comprising hydrophilic blocks and at least one fatty chain, and copolymers of PEG-180, of tetramethoxymethylglycouril and of Laureth-50 or octoxynol-40.

16. Composition according to claim 11, in which the associative polymers of cationic type are chosen from the group formed by cationic associative polyurethanes, quaternized cellulose derivatives comprising at least one fatty chain, and cationic polyvinyllactams.

17. Composition according to claim 1, in which the associative polymer is present in a content ranging from 0.01% to 30% by weight, of associative polymer(s) relative to the total weight of the composition.

18. Composition according to claim 1, in which the associative polymer is present in a content ranging from 0.05% to 15% by weight, of associative polymer(s) relative to the total weight of the composition.

19. Composition according to claim 1, in which the said composition comprises at least one fatty chain.

20. Composition according to the claim 19, in which the said fatty phase contains at least one fatty substance that is liquid at room temperature and at atmospheric pressure and/or at least one fatty substance that is solid at room temperature and at atmospheric pressure.

21. Composition according to claim 1, in which the said composition is in a fluid form of vaporizable or non-vaporizable liquid type, or in the form of a paste, a direct or inverse emulsion, a gel or an impregnated support.

22. Composition according to claim 1, in which the said composition is in a solid form, especially a compact, pulverulent or cast form, or in the form of a stick.

23. Composition according to claim 1, in which the said composition is in the form of a care product, an antisun product, an after-sun product, a daily photoprotective care product, a body care product, a foundation to be applied to the face or the neck, a concealer product, a complexion corrector, a tinted cream, a facial makeup base or a body makeup composition.

24. Non-therapeutic process for treating keratin materials and/or fibres, comprising at least the step of applying to the said keratin materials and/or fibres at least one coat of a cosmetic composition as defined according to claim 1.
